(19) United States
(12) Patent Application Publication
(10) Pub. No.: US 2026/0279593 A1
(43) Pub. Date: Sep. 17, 2026

(54) IDENTIFYING, QUANTIFYING, AND PHENOTYPING VASCULAR CALCIFICATION IN PATIENTS

(71) Applicant: UNIVERSITY OF PITTSBURGH - OF THE COMMONWEALTH SYSTEM OF HIGHER EDUCATION, Pittsburgh, PA (US)

(72) Inventors: Mehdi Ramezanpour Koomleh, Pittsburgh, PA (US); Anne M. Robertson, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/668,456

(22) Filed: May 5, 2026

Related U.S. Application Data
(63) Continuation of application No. PCT/US2025/010448, filed on Jan. 6, 2025.
(60) Provisional application No. 63/620,135, filed on Jan. 11, 2024.

(51) Int. Cl. G16H 50/30; G06T 7/11; G16H 10/60; G16H 15/00; G16H 20/10; G16H 30/40

(52) U.S. Cl. CPC G16H 50/30; G06T 7/11; G16H 10/60; G16H 15/00; G16H 20/10; G16H 30/40; G06T 2207/10072; G06T 2207/30101

(57) ABSTRACT

A method for identifying, quantifying, and phenotyping vascular calcification (VC) in patients includes accessing a set of medical scan images of a vascular tissue sample extracted from the one or more patients, and further inputting the set of medical scan images into one or more machine-learning models trained to segment the set of medical scan images to identify a tissue region, a lipid pool region, and a set of vascular calcifications detectable from the set of medical scan images. The method further includes determining, based on the segmented set of medical scan images, one or more feature characteristics corresponding to each of the identified set of vascular calcifications, and identifying each of the identified set of vascular calcifications as corresponding to one or more of a plurality of vascular calcification phenotypes based at least in part on the one or more feature characteristics and the identified lipid pool region.

### CROSS-REFERENCE TO RELATED APPLICATIONS

This application is a continuation of International Application No. PCT/US2025/010448, filed Jan. 6, 2025, which claims the benefit under 35 U.S.C. § 119(e) of U.S. Provisional Patent Application No. 63/620,135, filed Jan. 11, 2024, the contents of which are incorporated herein by reference in their entirety, and to which priority is claimed.

### STATEMENT REGARDING FEDERALLY-SPONSORED RESEARCH

This invention was made with government support under NS097457 awarded by the National Institutes of Health. The government has certain rights in the invention.

### TECHNICAL FIELD

This application relates generally to vascular calcification, and, more particularly, to identifying, quantifying, and phenotyping vascular calcification in patients.

### BACKGROUND

Vascular calcification (VC) generally includes mineral depositions of calcium-phosphate complexes that may accumulate in a patient's vasculature. While some VC accumulation may be a part of normal bodily functions as patients age, certain diseases, such as diabetes, hypertension, and chronic kidney disease (CKD) may often precipitate or exacerbate VC. Indeed, vascular calcifications may be linked to major adverse cardiovascular events (MACE). For example, vascular calcifications in fibrous caps within atherosclerotic plaques may adversely impact cap vulnerability. The rupture of these fibrous caps may release the plaque contents into the bloodstream, resulting in the formation of thrombi. Thrombi may obstruct blood flow, and thus lead to a MACE, such as an ischemic stroke, myocardial infarction (MI), or congestive heart failure (CHF).

In some instances, vascular calcifications may exhibit clinically-significant phenotypes that may either impede the failure of certain calcified vascular tissues or otherwise precipitate the failure of certain calcified vascular tissues. As an example, some macrocalcifications may stabilize plaques in coronary arteries, and thus may provide protection against potential calcified vascular tissue failure. As an example to the contrary, certain macrocalcifications with jagged edges may engender regions of stress concentrations in cerebral aneurysms or other similar vascular bulges, for example, and thus precipitate potential calcified vascular tissue failure. In other words, based on their respective topologies, macrocalcifications may be either promotive of patient health or detrimental to patient health.

In similar instances, microcalcifications may induce stress concentrations in the surrounding area of calcified vascular tissues. Additionally, high densities of microcalcifications may be associated with low levels of collagen fibers, which may lower the load-bearing capacity of calcified vascular tissues. Thus, microcalcifications may generally be detrimental to patient health. Furthermore, potential failure of calcified vascular tissues may be strongly linked to the environment neighboring the calcified vascular tissue. For example, in instances in which calcifications are embedded in lipid pools (e.g., atherosclerotic calcifications), potential failure of the calcified vascular tissue may be more gradual and allow for repair of the calcified vascular tissue. In contrast, in instances in which calcifications are not embedded in lipid pools (e.g., non-atherosclerotic calcifications), failure of the calcified vascular tissue may be abrupt and decisive. It may be thus useful to provide techniques to identify, quantify, and phenotype vascular calcification in patients.

### SUMMARY

Embodiments of the present disclosure are directed toward one or more computing devices, methods, and non-transitory computer-readable media for identifying, quantifying, and phenotyping vascular calcification (VC) in one or more patients. In certain embodiments, one or more computing devices may access a set of medical scan images (e.g., a set of micro computed tomography (μ-CT) images, micro positron emission tomography (μ-PET) images, micron single-photon emission computed tomography (u-SPECT) images, optical coherence tomography (OCT) images, optical coherence tomography angiography (OCT-A) images, and so forth) of a vascular tissue sample extracted from one or more patients. In certain embodiments, the one or more computing devices may then input the set of medical scan images into one or more machine-learning models (e.g., a segmentation and classification machine-learning pipeline, image thresholding model, and unsupervised clustering model) trained to segment the set of medical scan images to identify a tissue region, a lipid pool region, and a set of vascular calcifications detectable from the set of medical scan images.

For example, in certain embodiments, the one or more machine-learning models may include a semantic segmentation model, a tissue sample classification model (e.g., neural network classifier), a lipid pool classification model (e.g., neural network classifier), an image thresholding model, and a downstream unsupervised clustering model. In certain embodiments, the semantic segmentation model (e.g., U-Net or other similar semantic segmentation model) may be trained to segment the set of medical scan images to generate a first two-dimensional (2D) segmentation map including a first set of pixels corresponding to tissue regions and a second set of pixels corresponding to background regions. In certain embodiments, the one or more computing devices may then flatten the first 2D segmentation map into a feature map, and then the feature map, a set of pixel spatial information, and a set of pixel color intensity information associated with the set of medical scan images may be inputted into the tissue sample classification model (e.g., neural network classifier).

In certain embodiments, the tissue sample classification model (e.g., neural network classifier) may then identify one or more intra-slice tissue regions detectable from the first set of pixels corresponding to tissue regions in the first 2D segmentation map and generate a second 2D segmentation map including the first set of pixels corresponding to tissue regions, the second set of pixels corresponding to background regions, and a third set of pixels associated with the first set of pixels and corresponding to the identified one or more intra-slice tissue regions. In certain embodiments, the one or more computing devices may then flatten the second 2D segmentation map into a one-dimensional (1D) feature map and extract one or more pixels of the first set of pixels corresponding to tissue regions by passing the 1D feature map through a foreground-pass filter and extract one or more pixels of the second set of pixels corresponding to background regions by passing the 1D feature map through a background-pass filter.

In certain embodiments, the one or more computing devices may then input the extracted one or more pixels of the first set of pixels corresponding to tissue regions and the set of pixel spatial information into the lipid pool classification model (e.g., neural network classifier) to generate a prediction of a class label for one or more lipid pool regions detectable at least in part from the extracted one or more pixels and the set of pixel spatial information. In certain embodiments, the one or more computing devices may then combine the predicted class label for the one or more lipid pool regions, the extracted one or more pixels of the first set of pixels corresponding to tissue regions, and the extracted one or more pixels of the second set of pixels corresponding to background regions and the second 2D segmentation map may be then updated based thereon. Thus, the one or more computing devices may generate the updated second 2D segmentation map, which includes the first set of pixels corresponding to background regions, the second set of pixels corresponding to the identified one or more intra-slice tissue regions, the third set of pixels corresponding to tissue regions and the predicted class label for the one or more lipid pool regions.

In certain embodiments, upon generating the updated second 2D segmentation map, the one or more computing devices may then determine one or more feature characteristics corresponding to each of an identified set of vascular calcifications detectable from the set of medical scan images (e.g., μ-CT images, μ-PET images, μ-SPECT images, OCT images, OCT-A images, and so forth). For example, in certain embodiments, the one or more computing devices may identify a set of vascular calcifications present within the set of medical scan images by further inputting the set of medical scan images into an image thresholding model to identify the set of vascular calcifications based on, for example, a predetermined pixel intensity threshold value. In certain embodiments, the one or more computing devices may then determine one or more feature characteristics of the identified set of vascular calcifications. In certain embodiments, the one or more feature characteristics of the identified set of vascular calcifications may include, for example, one or more of a size, a spatial distribution, a topology, a porosity (sparsity), or a lipid pool colocalization for each of the identified set of vascular calcifications.

In certain embodiments, the one or more computing devices may then identify each of the identified set of vascular calcifications as corresponding to one or more of a number of vascular calcification phenotypes based on the one or more feature characteristics and the identified lipid pool region. For example, in certain embodiments, the one or more computing devices may identify each of the identified set of vascular calcifications as corresponding to one or more of a number of vascular calcification phenotypes utilizing, for example, an unsupervised clustering model (e.g., a density-based spatial clustering of applications with noise (DBSCAN) clustering model) or other similar clustering model (e.g., k-means clustering model, hierarchical clustering model) to cluster similar vascular calcifications of the identified set of vascular calcifications into one or more predetermined phenotype classes. For example, in one embodiment, the vascular calcification phenotypes may include a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall (e.g., innermost layer, middle layer, and outermost layer).

Thus, in accordance with the presently disclosed embodiments, one or more computing devices, methods, and non-transitory computer-readable media may be provided for identifying, quantifying, and phenotyping vascular calcification (VC) in one or more patients. Indeed, by providing a segmentation and classification machine-learning pipeline, image thresholding model, and downstream unsupervised clustering model suitable for accurately identifying, quantifying, and phenotyping vascular calcifications in one or more patients, the present embodiments may accurately and compute-efficiently identify one or more clinically-significant biomarkers for assessing and characterizing a patient's risk for a major adverse cardiovascular event (MACE), such as myocardial infarction (MI), acute MI, ischemic stroke, hemorrhagic stroke, congestive heart failure (CHF), and so forth.

Specifically, by accurately identifying, quantifying, and phenotyping vascular calcifications in accordance with the presently disclosed embodiments, the vascular calcifications (e.g., macrocalcifications, atherosclerotic calcifications) characterized as being potentially promotive of patient health and the vascular calcifications (e.g., microcalcifications, non-atherosclerotic calcifications) characterized as being potentially detrimental to patient health may be identified in a noninvasive and compute-efficient manner. Additionally, the provided segmentation and classification machine-learning pipeline, image thresholding model, and unsupervised clustering model may be further suitable for utilizing the identified, quantified, and phenotyped vascular calcifications to predict disease progression, disease regression, and patient treatment response in accordance with the presently disclosed embodiments.

The present embodiments described herein may further provide a number of technical improvements to the functioning of computing systems. For example, in some embodiments, the implementation of the one or more machine-learning models (e.g., a segmentation and classification machine-learning model pipeline, an image thresholding model, and an unsupervised clustering model) may be memory-efficient and compute-efficient in that while the semantic segmentation model (e.g., U-Net model) may be leveraged and trained on a sparsely annotated data set of medical scan images (e.g., μ-CT images, μ-PET images, μ-SPECT images, OCT images, OCT-A images, and so forth), the 2D segmentation map generated by the semantic segmentation model (e.g., U-Net model) may be flattened into one or more 1D feature maps and utilized to train the tissue sample classification model (e.g., neural network classifier) and the lipid pool classification model (e.g., neural network classifier) without having to perform again a feature extraction process on the data set of medical scan images.

Specifically, the present embodiments may include a unique feature-representation transfer learning process that is able to leverage the fact the extracted features utilized to identify lipid pool pixel regions may often overlap the extracted features utilized to segment and identify the tissue sample pixel regions. Thus, the present unique feature-representation transfer learning process may reduce the number of pixels involved in the training of at least the lipid pool classification model (e.g., neural network classifier). Indeed, by utilizing this unique feature-representation transfer learning process and reducing the number of pixels involved in the training of at least the lipid pool classification model (e.g., neural network classifier), compute-intensive and memory-intensive processing workloads associated with loading and processing the voluminous pixels in high-resolution medical scan images (e.g., u-CT images, μ-PET images, μ-SPECT images, OCT images, OCT-A images, and so forth) may be markedly reduced. In this way, overall processing device (e.g., CPU, GPU, or AI accelerator) performance in terms of execution time, latency, power consumption, and clock speed may all be markedly improved.

Among the provided embodiments are:

A method for identifying, quantifying, and phenotyping vascular calcification (VC) in

one or more patients, comprising, by one or more computing devices: accessing a set of medical scan images of a vascular tissue sample extracted from the one or more patients; inputting the set of medical scan images into one or more machine-learning models trained to segment the set of medical scan images to identify a tissue region, a lipid pool region, and a set of vascular calcifications detectable from the set of medical scan images; determining, based on the segmented set of medical scan images, one or more feature characteristics corresponding to each of the identified set of vascular calcifications; and identifying each of the identified set of vascular calcifications as corresponding to one or more of a plurality of vascular calcification phenotypes based at least in part on the one or more feature characteristics and the identified lipid pool region.

The plurality of vascular calcification phenotypes comprises a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall (e.g., innermost layer, middle layer, and outermost layer).

Identifying each of the identified set of vascular calcifications as corresponding to one or more of the plurality of vascular calcification phenotypes comprises identifying one or more biomarkers associated with a risk for a major adverse cardiovascular event (MACE).

Identifying each of the identified set of vascular calcifications as corresponding to one or more of the plurality of vascular calcification phenotypes comprises identifying a risk for a major adverse cardiovascular event (MACE) for the one or more patients.

Identifying each of the identified set of vascular calcifications as corresponding to one or more of the plurality of vascular calcification phenotypes comprises identifying each of the identified set of vascular calcifications as corresponding to one of a destabilizing vascular calcification or a protective vascular calcification.

Identifying each of the identified set of vascular calcifications as corresponding to at least one of the plurality of vascular calcification phenotypes based at least in part on whether each of the identified set of vascular calcifications is associated with the identified lipid pool region.

Determining the one or more feature characteristics comprises determining one or more of a size, a spatial distribution, a topology, a porosity (sparsity), or a lipid pool colocalization for each of the identified set of vascular calcifications.

The one or more machine-learning models comprise a semantic segmentation model.

The semantic segmentation model comprises a U-Net architecture.

The one or more machine-learning models comprise the semantic segmentation model and at least one classification model.

The at least one classification model comprises one or more of a convolutional neural network (CNN), a deep convolutional neural network (DCNN), a fully connected neural network (FCNN), or a fully convolutional network (FCN).

Training the semantic segmentation model includes accessing a data set of medical scan images of one or more vascular tissue samples extracted from a plurality of patients, wherein the data set of medical scan images comprises sparse annotations of tissue regions and lipid pool regions in the one or more vascular tissue samples; partitioning the data set of medical scan images into a model-training data set and a model-validation data set; training, based on the model-training data set, the semantic segmentation model to generate a segmentation map comprising a first set of pixels corresponding to tissue regions and a second set of pixels corresponding to background regions; and evaluating the semantic segmentation model based on the model-validation data set.

The at least one classification model comprises a first classification model, the method further comprising: training the first classification model by: accessing the segmentation map generated by the semantic segmentation model, a set of pixel spatial information, and a set of pixel color intensity information associated with the data set of medical scan images; flattening the segmentation map into a feature map; inputting the feature map, the set of pixel spatial information, and the set of pixel color intensity information into the first classification model to identify one or more intra-slice tissue regions detectable at least in part from the first set of pixels corresponding to tissue regions in the segmentation map; and outputting, by the first classification model, a second segmentation map, the second segmentation map comprising the first set of pixels corresponding to tissue regions and the second set of pixels corresponding to background regions.

The one or more machine-learning models comprise a second classification model associated with the first classification model, the method further comprising: training the second classification model by: manual extraction of feature maps by passing the input images through convolutional layers with predefined weights; flattening the generated feature maps into one-dimensional feature maps; extracting one or more pixels of the obtained feature maps corresponding to tissue regions by passing the one-dimensional feature maps through a foreground-pass filter; inputting the extracted one or more pixels and the set of pixel spatial information into the second classification model to generate a prediction of a class label for one or more lipid pool regions detectable at least in part from the extracted one or more pixels and the set of pixel spatial information; and outputting, by the second classification model, the prediction of the class label for the one or more lipid pool regions.

The one or more machine-learning models comprise an unsupervised clustering model.

The unsupervised clustering model comprises one or more of a k-means clustering model, a hierarchical clustering model, or a density-based spatial clustering of applications with noise (DBSCAN) clustering model.

The unsupervised clustering model is trained to identify a plurality of clusters of the identified set of vascular calcifications as each corresponding to one or more of the plurality of vascular calcification phenotypes.

The set of medical scan images comprises one or more micro computed tomography (μ-CT) images, one or more micro positron emission tomography (μ-PET) images, one or more micron single-photon emission computed tomography (u-SPECT) images, one or more optical coherence tomography (OCT) images, one or more optical coherence tomography angiography (OCT-A) images, or any combination thereof.

The set of medical scans comprises a first set of medical scans of a first vascular tissue sample extracted from the one or more patients at an initial date, the method further comprising: accessing a second set of medical scan images of a second vascular tissue sample extracted from the one or more patients, the second vascular tissue sample being extracted from the one or more patients at a date subsequent to the initial date; inputting the second set of medical scan images into the one or more machine-learning models trained to segment the second set of medical scan images to identify a second tissue region, a second lipid pool region, and a second set of vascular calcifications detectable from the second set of medical scan images; determining, based on the segmented second set of medical scan images, one or more second featural characteristics corresponding to each of the identified second set of vascular calcifications; identifying each of the identified second set of vascular calcifications as corresponding to one or more of the plurality of vascular calcification phenotypes based at least in part on the one or more second featural characteristics and the identified second lipid pool region; and estimating, based on the vascular calcifications identified as corresponding to one or more of the plurality of vascular calcification phenotypes, a progression of VC or a regression of VC in the one or more patients.

The method further comprising estimating, based on the vascular calcifications identified as corresponding to one or more of the plurality of vascular calcification phenotypes, whether the one or more patients is responsive to a treatment.

The treatment comprises one or more calcification inhibitors.

The method further comprising identifying an effective treatment regimen of one or more calcification inhibitors to treat the one or more patients based on the estimated progression of VC or regression of VC.

The date subsequent to the initial date comprises one or more dates selected from the group comprising approximately 0.25 months, 0.5 months, 0.75 months, 1 month, 3 months, 6 months, 9 months, 12 months, 15 months, 18 months, 21 months, 24 months, 27 months, 30 months, 33 months, 36 months, 39 months, 42 months, 45 months, or 48 months from the initial date.

The method further comprising: generating a report based on the vascular calcifications of the identified set of vascular calcifications identified as corresponding to one or more of the plurality of vascular calcification phenotypes; and transmitting the report to a computing device associated with a clinician or a scientist.

One or more computer-readable non-transitory storage media embodying software that is operable, when executed by one or more processors of a computing system, to perform the steps of any of paragraphs [0016] through [0040].

A computing system, comprising: one or more processors; and a non-transitory memory coupled to the one or more processors and comprising instructions executable by the one or more processors, the one or more processors operable when executing the instructions to perform the steps of any of paragraphs [0016] through [0040].

### BRIEF DESCRIPTION OF THE DRAWINGS

One or more drawings included herein are in color in accordance with 37 CFR § 1.84. The color drawings are necessary to illustrate the invention.

FIG. 1 illustrates a clinical and computing environment in accordance with some embodiments disclosed herein.

FIG. 2 illustrates an example embodiment of a segmentation and classification machine-learning pipeline suitable for segmenting and classifying vascular tissue samples and lipid pool regions in medical scans in accordance with some embodiments disclosed herein.

FIGS. 3A-3C illustrate one or more example renderings for identifying, quantifying, and phenotyping vascular calcification (VC) in one or more patients in accordance with some embodiments disclosed herein.

FIG. 4 illustrates a flow diagram of a method for identifying, quantifying, and phenotyping vascular calcification (VC) in one or more patients in accordance with some embodiments disclosed herein.

FIG. 5 illustrates a flow diagram of a method for estimating a progression of vascular calcification (VC) or regression of VC in one or more patients in accordance with some embodiments disclosed herein.

FIG. 6 illustrates an example computing system in accordance with some embodiments disclosed herein.

FIG. 7 illustrates a diagram of an example artificial intelligence (AI) architecture included as part of the example computing system of FIG. 6 in accordance with some embodiments disclosed herein.

### DESCRIPTION OF EXAMPLE EMBODIMENTS

Embodiments of the present disclosure are directed toward one or more computing devices, methods, and non-transitory computer-readable media for identifying, quantifying, and phenotyping vascular calcification (VC) in one or more patients. In certain embodiments, one or more computing devices may access a set of medical scan images (e.g., a set of micro computed tomography (μ-CT) images, micro positron emission tomography (μ-PET) images, micron single-photon emission computed tomography (u-SPECT) images, optical coherence tomography (OCT) images, optical coherence tomography angiography (OCT-A) images, and so forth) of a vascular tissue sample extracted from one or more patients. In certain embodiments, the one or more computing devices may then input the set of medical scan images into one or more machine-learning models (e.g., a segmentation and classification machine-learning pipeline, image thresholding model, and unsupervised clustering model) trained to segment the set of medical scan images to identify a tissue region, a lipid pool region, and a set of vascular calcifications detectable from the set of medical scan images.

For example, in certain embodiments, the one or more machine-learning models may include a semantic segmentation model, a tissue sample classification model (e.g., neural network classifier), a lipid pool classification model (e.g., neural network classifier), an image thresholding model, and a downstream unsupervised clustering model. In certain embodiments, the semantic segmentation model (e.g., U-Net or other similar semantic segmentation model) may be trained to segment the set of medical scan images to generate a first two-dimensional (2D) segmentation map including a first set of pixels corresponding to tissue regions and a second set of pixels corresponding to background regions. In certain embodiments, the one or more computing devices may then flatten the first 2D segmentation map into a feature map, and then the feature map, a set of pixel spatial information, and a set of pixel color intensity information associated with the set of medical scan images may be inputted into the tissue sample classification model (e.g., neural network classifier).

In certain embodiments, the tissue sample classification model (e.g., neural network classifier) may then identify one or more intra-slice tissue regions detectable from the first set of pixels corresponding to tissue regions in the first 2D segmentation map and generate a second 2D segmentation map including the first set of pixels corresponding to tissue regions, the second set of pixels corresponding to background regions, and a third set of pixels associated with the first set of pixels and corresponding to the identified one or more intra-slice tissue regions. In certain embodiments, the one or more computing devices may then flatten the second 2D segmentation map into a one-dimensional (1D) feature map and extract one or more pixels of the first set of pixels corresponding to tissue regions by passing the 1D feature map through a foreground-pass filter and extract one or more pixels of the second set of pixels corresponding to background regions by passing the 1D feature map through a background-pass filter.

In certain embodiments, the one or more computing devices may then filter a set of manually extracted features that are generated by passing the input images through convolutional layers with predefined weights using the foreground-pass filter, and input the passed pixel information along with their spatial information into the lipid pool classification model (e.g., neural network classifier) to generate a prediction of a class label for one or more lipid pool regions detectable at least in part from the extracted one or more pixels and the set of pixel spatial information. In certain embodiments, the one or more computing devices may then combine the predicted class label for the one or more lipid pool regions, the extracted one or more pixels of the first set of pixels corresponding to tissue regions, and the extracted one or more pixels of the second set of pixels corresponding to background regions and the second 2D segmentation map may be then updated based thereon. Thus, the one or more computing devices may generate the updated second 2D segmentation map, which includes the first set of pixels corresponding to tissue regions, the second set of pixels corresponding to background regions, the third set of pixels corresponding to the identified one or more intra-slice tissue regions, and the predicted class label for the one or more lipid pool regions.

In certain embodiments, upon generating the updated second 2D segmentation map, the one or more computing devices may then determine one or more feature characteristics corresponding to each of an identified set of vascular calcifications detectable from the set of medical scan images (e.g., μ-CT images, μ-PET images, μ-SPECT images, OCT images, OCT-A images, and so forth). For example, in certain embodiments, the one or more computing devices may identify a set of vascular calcifications present within the set of medical scan images by further inputting the set of medical scan images into an image thresholding model to identify the set of vascular calcifications based on, for example, a predetermined pixel intensity threshold value. In certain embodiments, the one or more computing devices may then determine one or more feature characteristics of the identified set of vascular calcifications. In certain embodiments, the one or more feature characteristics of the identified set of vascular calcifications may include, for example, one or more of a size, a spatial distribution, a topology, a porosity, or a lipid pool colocalization for each of the identified set of vascular calcifications.

In certain embodiments, the one or more computing devices may then identify each of the identified set of vascular calcifications as corresponding to one or more of a number of vascular calcification phenotypes based on the one or more feature characteristics and the identified lipid pool region. For example, in certain embodiments, the one or more computing devices may identify each of the identified set of vascular calcifications as corresponding to one or more of a number of vascular calcification phenotypes utilizing, for example, an unsupervised clustering model (e.g., a density-based spatial clustering of applications with noise (DBSCAN) clustering model) or other similar clustering model (e.g., k-means clustering model, hierarchical clustering model) to cluster similar vascular calcifications of the identified set of vascular calcifications into one or more predetermined phenotype classes. For example, in one embodiment, the vascular calcification phenotypes may include a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall (e.g., innermost layer, middle layer, and outermost layer).

Thus, in accordance with the presently disclosed embodiments, one or more computing devices, methods, and non-transitory computer-readable media may be provided for identifying, quantifying, and phenotyping vascular calcification (VC) in one or more patients. Indeed, by providing a segmentation and classification machine-learning pipeline, image thresholding model, and downstream unsupervised clustering model suitable for accurately identifying, quantifying, and phenotyping vascular calcifications in one or more patients, the present embodiments may accurately and compute-efficiently identify one or more clinically-significant biomarkers for assessing and characterizing a patient's risk for a major adverse cardiovascular event (MACE), such as myocardial infarction (MI), acute MI, ischemic stroke, hemorrhagic stroke, congestive heart failure (CHF), and so forth.

Specifically, by accurately identifying, quantifying, and phenotyping vascular calcifications in accordance with the presently disclosed embodiments, the vascular calcifications (e.g., macrocalcifications, atherosclerotic calcifications) characterized as being potentially promotive of patient health and the vascular calcifications (e.g., microcalcifications, non-atherosclerotic calcifications) characterized as being potentially detrimental to patient health may be identified in a noninvasive and compute-efficient manner. Additionally, the provided segmentation and classification machine-learning pipeline, image thresholding model, and unsupervised clustering model may be further suitable for utilizing the identified, quantified, and phenotyped vascular calcifications to predict disease progression, disease regression, and patient treatment response in accordance with the presently disclosed embodiments.

The present embodiments described herein may further provide a number of technical improvements to the functioning of computing systems. For example, in some embodiments, the implementation of the one or more machine-learning models (e.g., a segmentation and classification machine-learning model pipeline, an image thresholding model, and an unsupervised clustering model) may be memory-efficient and compute-efficient in that while the semantic segmentation model (e.g., U-Net model) may be leveraged and trained on a sparsely annotated data set of medical scan images (e.g., μ-CT images, μ-PET images, μ-SPECT images, OCT images, OCT-A images, and so forth), the 2D segmentation map generated by the semantic segmentation model (e.g., U-Net model) may be flattened into one or more 1D feature maps and utilized to train the tissue sample classification model (e.g., neural network classifier) and the lipid pool classification model (e.g., neural network classifier) without having to perform again a feature extraction process on the data set of medical scan images.

Specifically, the present embodiments may include a unique feature-representation transfer learning process that is able to leverage the fact the extracted features utilized to identify lipid pool pixel regions may often overlap the extracted features utilized to segment and identify the tissue sample pixel regions. Thus, the present unique feature-representation transfer learning process may reduce the number of pixels involved in the training of at least the lipid pool classification model (e.g., neural network classifier). Indeed, by utilizing this unique feature-representation transfer learning process and reducing the number of pixels involved in the training of at least the lipid pool classification model (e.g., neural network classifier), compute-intensive and memory-intensive processing workloads associated with loading and processing the voluminous pixels in high-resolution medical scan images (e.g., u-CT images, μ-PET images, μ-SPECT images, OCT images, OCT-A images, and so forth) may be markedly reduced. In this way, overall processing device (e.g., CPU, GPU, or AI accelerator) performance in terms of execution time, latency, power consumption, and clock speed may all be markedly improved.

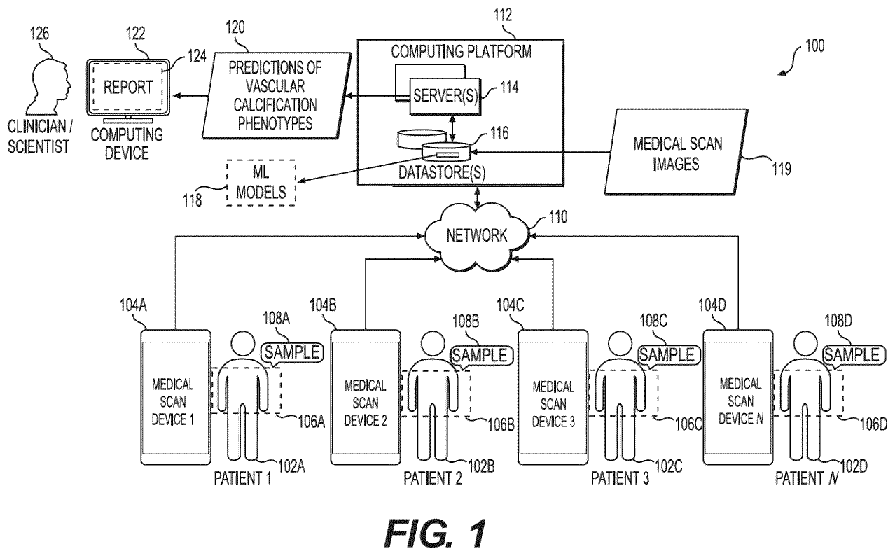

FIG. 1 illustrates an example embodiment of a clinical and computing environment 100 that may be utilized to identify, quantify, and phenotype vascular calcification (VC) in one or more patients, in accordance with the presently disclosed embodiments. As depicted, the clinical and computing environment 100 may include a number of patients 102A (e.g., “Patient 1”), 102B (e.g., “Patient 2”), 102C (e.g., “Patient 3”), and 102D (e.g., “Patient N”) each associated with medical scan devices 104A (e.g., “Medical Scan Device 1”), 104B (e.g., “Medical Scan Device 2”), 104C (e.g., “Medical Scan Device 3”), and 104D (e.g., “Medical Scan Device N”) that may be suitable for capturing medical scans 106A, 106B, 106C, and 106D of vascular tissue samples 108A, 108B, 108C, and 108D.

In certain embodiments, the vascular tissue samples 108A, 108B, 108C, and 108D may be extracted from the number of patients 102A, 102B, 102C, and 102D in vivo, for example, during a patient visit to a clinical setting (e.g., a clinic, a hospital, an outpatient facility, and so forth). or during a clinical study or a clinical trial in which the number of patients 102A, 102B, 102C, and 102D may be participants. In one embodiment, the number of patients 102A, 102B, 102C, and 102D may each include a patient potentially at risk for a MACE, such as a patient having one or more of diabetes, hypertension, chronic kidney disease (CKD), or other similar disease leading to, or exacerbating the calcification of a patient's vasculature. In another embodiment, the number of patients 102A, 102B, 102C, and 102D may each include a patient having once before experienced a MACE, such as a patient having once experienced an ischemic stroke, myocardial infarction (MI), or congestive heart failure (CHF). In another embodiment, it should be appreciated that the number of patients 102A, 102B, 102C, and 102D may each include any subject, such as one or more living humans, one or more human cadavers, one or more living animals, or one or more animal cadavers.

In certain embodiments, the medical scan devices 104A, 104B, 104C, and 104D may each include one or more noninvasive vascular tissue sample capturing devices (e.g., one or more medical scanners), which may scan the vascular tissue samples 108A, 108B, 108C, and 108D and generate a set of high-resolution, 2D or 3D medical scans 106A, 106B, 106C, and 106D. In certain embodiments, the medical scans 106A, 106B, 106C, and 106D may each include, for example, one or more micro computed tomography (μ-CT) scans, one or more micro positron emission tomography (μ-PET) scans, one or more micron single-photon emission computed tomography (u-SPECT) scans, one or more optical coherence tomography (OCT) scans, one or more optical coherence tomography angiography (OCT-A) scans, or other similar medical scans suitable for capturing and rendering 2D cross-sectional images of the vascular tissue samples 108A, 108B, 108C, and 108D in a slice-by-slice manner.

In certain embodiments, as further depicted by FIG. 1, the medical scan devices 104A, 104B, 104C, and 104D may be coupled to a computing platform 112 via one or more communication network(s) 110. In certain embodiments, the computing platform 112 may include, for example, a cloud-based computing architecture suitable for hosting and executing one or more machine-learning models 118 that may be trained to identify, quantify, and phenotype vascular calcifications in one or more of the number of patients 102A, 102B, 102C, and 102D in accordance with the presently disclosed embodiments. For example, in one embodiment, the computing platform 112 may include a Platform as a Service (PaaS) architecture, a Software as a Service (SaaS) architecture, an Infrastructure as a Service (IaaS) architecture, a Compute as a Service (CaaS) architecture, a Data as a Service (DaaS) architecture, a Database as a Service (DBaaS) architecture, or other similar cloud-based computing architecture (e.g., “X” as a Service (XaaS)).

In certain embodiments, as further depicted by FIG. 1, the computing platform 112 may include one or more processing devices 114 (e.g., servers) and one or more data stores 116. For example, in some embodiments, the one or more processing devices 114 (e.g., servers) may include one or more general purpose processors, graphic processing units (GPUs), application-specific integrated circuits (ASICs), systems-on-chip (SoCs), microcontrollers, field-programmable gate arrays (FPGAs), central processing units (CPUs), application processors (APs), visual processing units (VPUs), neural processing units (NPUs), neural decision processors (NDPs), deep learning processors (DLPs), tensor processing units (TPUs), neuromorphic processing units (NPUs), or any of various other processing device(s) or artificial intelligence (AI) accelerators that may be suitable for inputting a data set of medical scan images 119 of the vascular tissue samples 108A, 108B, 108C, and 108D into the one or more machine-learning models 118 and executing the one or more machine-learning models 118 to generate one or more predictions based thereon. Similarly, the data stores 116 may include, for example, one or more internal databases that may be utilized to store the data set of medical scan images 119 and the one or more machine-learning models 118.

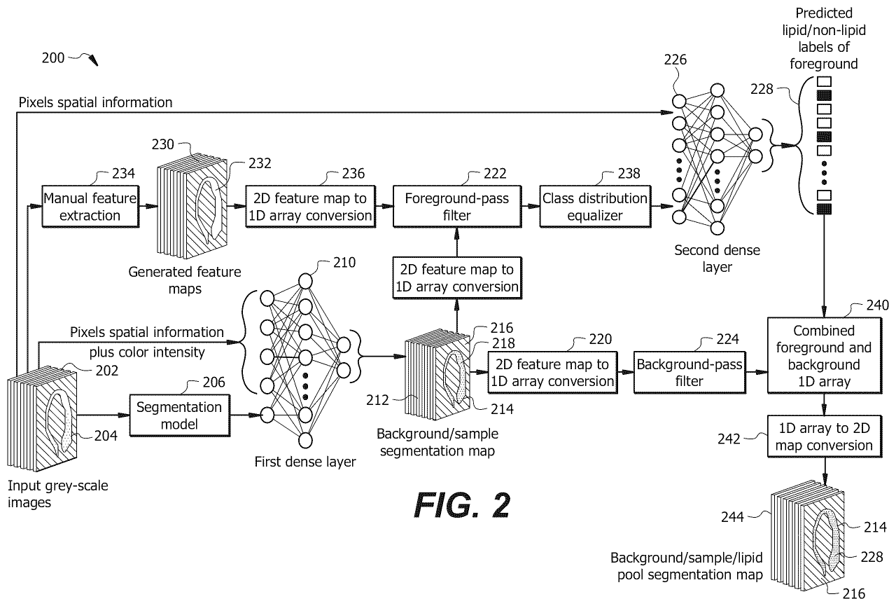

In certain embodiments, as will be discussed in greater detail below with respect to FIG. 2, upon the data stores 116 receiving and storing the data set of medical scan images 119, the one or more processing devices 114 (e.g., servers) may then access the data set of medical scan images 119 and execute the one or more machine-learning models 118 to generate one or more predictions of vascular calcification phenotypes 120 based on the data set of medical scan images 119. For example, in certain embodiments, the one or more processing devices 114 (e.g., servers) may load and execute the one or more machine-learning models 118 to identify, quantify, and phenotype vascular calcifications detectable from the data set of medical scan images 119 and generate and output the predictions of vascular calcification phenotypes 120. For example, in accordance with the presently disclosed embodiments, the predictions of vascular calcification phenotypes 120 may include predictions of one or more of a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, or a phenotype identifying a location of vascular calcifications within an arterial wall (e.g., innermost layer, middle layer, and outermost layer). Specifically, each of the preceding eight distinct phenotypes may be further classified according to their distribution across the arterial wall as innermost, middle, and outermost layers of the arterial wall.

In certain embodiments, as will be further discussed below with respect to FIG. 2, the one or more machine-learning models 118 may include a semantic segmentation model, a tissue sample classification model (e.g., neural network classifier), a lipid pool classification model (e.g., neural network classifier), an image thresholding model, and a downstream unsupervised clustering model collectively trained to generate and output the predictions of vascular calcification phenotypes 120. In one embodiment, one or more of the semantic segmentation model, the tissue sample classification model (e.g., neural network classifier), the lipid pool classification model (e.g., neural network classifier), the image thresholding model, and the unsupervised clustering model may be trained to generate and output the predictions of vascular calcification phenotypes 120 in accordance with a feature-representation transfer learning process or other transfer learning process (e.g., transference of pretrained weights) to generate and output the predictions of vascular calcification phenotypes 120. In another embodiment, the semantic segmentation model, the tissue sample classification model (e.g., neural network classifier), the lipid pool classification model (e.g., neural network classifier), the image thresholding model, and the unsupervised clustering model may be trained end-to-end to generate and output the predictions of vascular calcification phenotypes 120.

In certain embodiments, as further illustrated by FIG. 1, the one or more processing devices 114 (e.g., servers) may then transmit the predictions of vascular calcification phenotypes 120 to a computing device 122 and present a report 124 to a clinician or scientist 126 (e.g., a cardiologist, a cardiovascular invasive specialist, a cardiovascular scientist, a biomarker scientist, or a data scientist) that may be associated with one or more of the number of patients 102A, 102B, 102C, and 102D. In one embodiment, the report 124 may include a clinical report that may be associated with one or more of the number of patients 102A, 102B, 102C, and 102D to be provided and displayed, for example, to the clinician or scientist 126 (e.g., a cardiologist, a cardiovascular invasive specialist, a cardiovascular scientist, a biomarker scientist, or a data scientist) for purposes of research and/or the diagnosis, prognosis, and treatment of one or more of the number of patients 102A, 102B, 102C, and 102D. In another embodiment, the report 124 may include an interpretability and/or explainability report that may be associated with the one or more machine-learning models 118 to be provided and displayed, for example, to the clinician or scientist 126 (e.g., a cardiologist, a cardiovascular invasive specialist, a cardiovascular scientist, a biomarker scientist, or a data scientist) for purposes of ascertaining and elucidating the prediction and decision-making behaviors of the one or more machine-learning models 118.

FIG. 2 illustrates an example embodiment of a segmentation and classification machine-learning pipeline 200 suitable for segmenting and classifying vascular tissue samples and lipid pool regions in medical scans, in accordance with the presently disclosed embodiments. In one embodiment, the segmentation and classification machine-learning pipeline 200 may include a workflow process that may be implemented and executed by the one or more processing devices 114 (e.g., servers) of computing platform 112 as discussed above with respect to FIG. 1.

For example, referring to the segmentation and classification machine-learning pipeline 200, the one or more processing devices 114 (e.g., servers) may access a data set of medical scan images 202 of a vascular tissue sample 204. In one embodiment, the data set of medical scan images 202 may include one or more high-resolution μ-CT images, μ-PET images, μ-SPECT images, OCT images, or OCT-A images. In certain embodiments, the one or more processing devices 114 (e.g., servers) may then input the data set of medical scan images 202 into a segmentation model 206. For example, in certain embodiments, the segmentation model 206 may include a deep residual neural network (ResNet) image-classification network (e.g., ResNet-34, ResNet-50, ResNet-101, ResNet-152), a full-resolution residual network (FRRN), a fully convolutional network (FCN) (e.g., U-Net), a pyramid scene parsing network (PSPNet), a fully convolutional dense neural network (FCDenseNet), a multi-path refinement network (RefineNet), an atrous convolutional network (e.g., DeepLabV3, DeepLabV+), a semantic segmentation network (SegNet), or other semantic segmentation or instance segmentation model that may be suitable for generating a first 2D segmentation map identifying a first region of pixels corresponding to vascular tissue and a second region of pixels corresponding to background in the data set of medical scan images 202.

In certain embodiments, the one or more processing devices 114 (e.g., servers) may then flatten the first 2D segmentation map into a feature map, and then the feature map and a set of pixel spatial and pixel color intensity information associated with the data set of medical scan images 202 may be inputted into a tissue sample classification model 210. In certain embodiments, the tissue sample classification model 210 may include, for example, a convolutional neural network (CNN), a deep convolutional neural network (DCNN), a fully connected neural network (FCNN), or a fully convolutional network (FCN). In certain embodiments, the tissue sample classification model 210 may be utilized to identify intra-slice tissue regions detectable from the first region of pixels corresponding to tissue regions in the first 2D segmentation map and generate a second 2D segmentation map 212 including the first region of pixels 214 corresponding to tissue regions 218, the second region of pixels corresponding to background regions 216.

In certain embodiments, the one or more processing devices 114 (e.g., servers) may then flatten the second 2D segmentation map 212 into a 1D feature map 220 (e.g., a single vector) and extract one or more pixels of the first region of pixels corresponding to tissue regions 218 by passing the 1D feature map 220 (e.g., a single vector) through a foreground-pass filter 222 and extract one or more pixels of the second region of pixels corresponding to background regions 216 by passing the 1D feature map 220 (e.g., a single vector) through a background-pass filter 224. In certain embodiments, the foreground-pass filter 222 and the background-pass filter 224 may be utilized, for example, to extract respective foreground and background regions from the second 2D segmentation map 212 to facilitate lipid pool segmentation utilizing a lipid pool classification model 226 and ultimately to recover image information of the data set of medical scan images 202 after completing the tissue sample and lipid pool segmentation and classification.

In certain embodiments, the one or more processing devices 114 (e.g., servers) may then input the extracted one or more pixels of the first region of pixels corresponding to tissue regions 218 and the set of pixel spatial information into the lipid pool classification model 226 to generate a prediction of one or more class labels 228 for one or more lipid pool regions detectable from the extracted one or more pixels of the first region of pixels corresponding to tissue regions 218 and the set of pixel spatial information. Similar to the tissue sample classification model 210, the lipid pool classification model 226 may include, for example, a CNN, a DCNN, an FCNN, or an FCN. In certain embodiments, by leveraging the second 2D segmentation map 212 to extract the first region of pixels corresponding to tissue regions 218 and the second region of pixels corresponding to background regions 216, the total number of pixels involved in the training and execution of the lipid pool classification model 226 may be reduced due to these features having been already extracted upstream utilizing the segmentation model 206 and the tissue sample classification model 210.

Specifically, in one embodiment, the lipid pool classification model 226 may be trained in accordance with a feature-representation transfer learning process by leveraging the first region of pixels corresponding to tissue regions 218 and the second region of pixels corresponding to background regions 216 as generated by the segmentation model 206 and the tissue sample classification model 210. In another embodiment, the lipid pool classification model 226 may be trained based on a number of feature maps 230 that are extracted by passing the data set of medical scan images 202 through convolutional layers with predefined weights, for example. For example, in one embodiment, the data set of medical scans 202 (e.g., high-resolution μ-CT images, μ-PET images, μ-SPECT images, OCT images, or OCT-A images) may be annotated manually by drawing bounding geometries or contours, for example, representative of lipid pool regions and/or non-lipid pool regions.

In certain embodiments, 1D feature maps 236 (e.g., multiple vectors) may be generated based on the 2D feature maps 232. In certain embodiments, one or more pixels of a region of pixels corresponding, for example, to the feature maps 232 may be extracted by passing the 1D feature maps 236 (e.g., a single vector) through the foreground-pass filter 222. In one embodiment, if a significant class distribution imbalance is detected in the total number of pixels belonging to lipid pool regions across the training slices, resampling may be performed utilizing a class distribution equalizer 238.

In certain embodiments, as previously noted above, the one or more processing devices 114 (e.g., servers) may input an extracted one or more pixels of the first region of pixels corresponding to tissue regions 218, the manually extracted (e.g., using manual feature extraction 234) feature maps 230, and the set of pixel spatial information into the lipid pool classification model 226 to generate a prediction of one or more class labels 228 for lipid pool regions. In certain embodiments, the one or more processing devices 114 (e.g., servers) may then combine 240 the predicted one or more class labels 228 for the lipid pool regions, the extracted one or more pixels of the first region of pixels corresponding to tissue regions 218, and the extracted one or more pixels of the second region of pixels corresponding to background regions 216.

In certain embodiments, the one or more processing devices 114 (e.g., servers) may then convert 242 (e.g., upsample and reconstruct) the one-dimensional classification labels denoting background, tissue, and lipid pool regions to 2D segmentation maps, thereon. Thus, the one or more processing devices 114 (e.g., servers) may generate an updated second 2D segmentation map 244, which includes the first region of pixels corresponding to tissue regions 218, the second region of pixels corresponding to background regions 216, and the predicted one or more class labels 228 for the lipid pool regions.

In certain embodiments, although not illustrated with respect to FIG. 2, upon generating the updated second 2D segmentation map 244, the one or more processing devices 114 (e.g., servers) may then determine one or more feature characteristics corresponding to each of an identified set of vascular calcifications (e.g., vascular calcification particles) detectable from the data set of medical scan images 202 (e.g., μ-CT images, μ-PET images, μ-SPECT images, OCT images, OCT-A images, and so forth). For example, in certain embodiments, the one or more processing devices 114 (e.g., servers) may identify a set of vascular calcifications present within the data set of medical scan images 202 utilizing, for example, an image thresholding model (e.g., included as part of the one or more machine-learning models 118 discussed with respect to FIG. 1) to identify a set of vascular calcifications, and then determining one or more feature characteristics of the identified set of vascular calcifications.

For example, in one embodiment, the data set of medical scan images 202 may be inputted into the image thresholding model, which may be utilized to segment the data set of medical scan images 202 into a binary image of regions of background pixels (e.g., black or dark pixel regions) and regions of foreground pixels (e.g., white or bright pixel regions) corresponding to an identified set of vascular calcifications based on, for example, a predetermined pixel intensity threshold value. In certain embodiments, the one or more feature characteristics of the identified set of vascular calcifications may include, for example, one or more of a size, a spatial distribution, a topology, a porosity (sparsity), or a lipid pool colocalization for each of the identified set of vascular calcifications.

In certain embodiments, the one or more processing devices 114 (e.g., servers) may then identify each of the identified set of vascular calcifications as corresponding to one or more of a number of vascular calcification phenotypes based in part on the one or more feature characteristics. For example, in certain embodiments, the one or more processing devices 114 (e.g., servers) may identify each of the identified set of vascular calcifications as corresponding to one or more of a number of vascular calcification phenotypes utilizing, for example, an unsupervised clustering model (e.g., a density-based spatial clustering of applications with noise (DBSCAN) clustering model) or other similar clustering model (e.g., k-means clustering model, hierarchical clustering model) to cluster similar vascular calcifications of the identified set of vascular calcifications into one or more predetermined phenotype classes. For example, in one embodiment, the vascular calcification phenotypes may include a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall (e.g., innermost layer, middle layer, and outermost layer).

In certain embodiments, upon identifying the clusters of similar vascular calcifications of the identified set of vascular calcifications into one or more predetermined phenotype classes, the one or more processing devices 114 (e.g., servers) may estimate a progression of VC or a regression of VC in one or more of the number of patients 102A, 102B, 102C, and 102D. For example, in one embodiment, the one or more processing devices 114 (e.g., servers) may access an updated set of medical scan images of a vascular tissue sample extracted from one or more of the number of patients 102A, 102B, 102C, and 102D at a date subsequent to an initial date at which one or more of the vascular tissue samples 108A, 108B, 108C, and 108D are extracted. For example, the date subsequent to the initial date may include one or more dates selected from the group including approximately 0.25 months, 0.5 months, 0.75 months, 1 month, 3 months, 6 months, 9 months, 12 months, 15 months, 18 months, 21 months, 24 months, 27 months, 30 months, 33 months, 36 months, 39 months, 42 months, 45 months, or 48 months from the initial date.

In certain embodiments, the one or more processing devices 114 (e.g., servers) may also estimate whether one or more of the number of patients 102A, 102B, 102C, and 102D is responsive to a treatment based on the updated set of medical scan images. For example, in one embodiment, the treatment may include, for example, one or more calcification inhibitors. In certain embodiments, the one or more processing devices 114 (e.g., servers) may further identify an effective treatment regimen of one or more calcification inhibitors to treat one or more of the number of patients 102A, 102B, 102C, and 102D based on the estimated progression of VC or regression of VC.

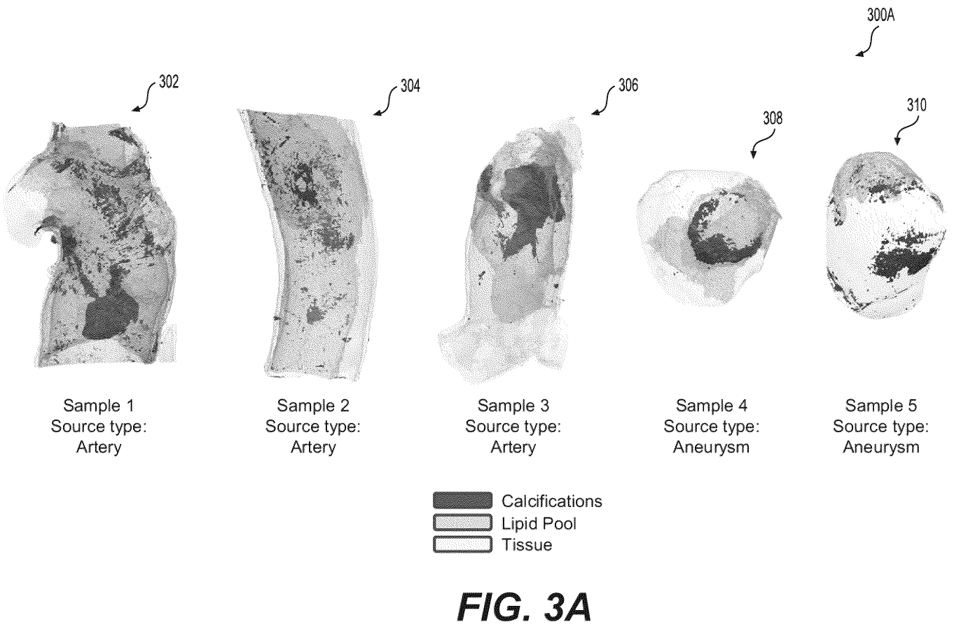

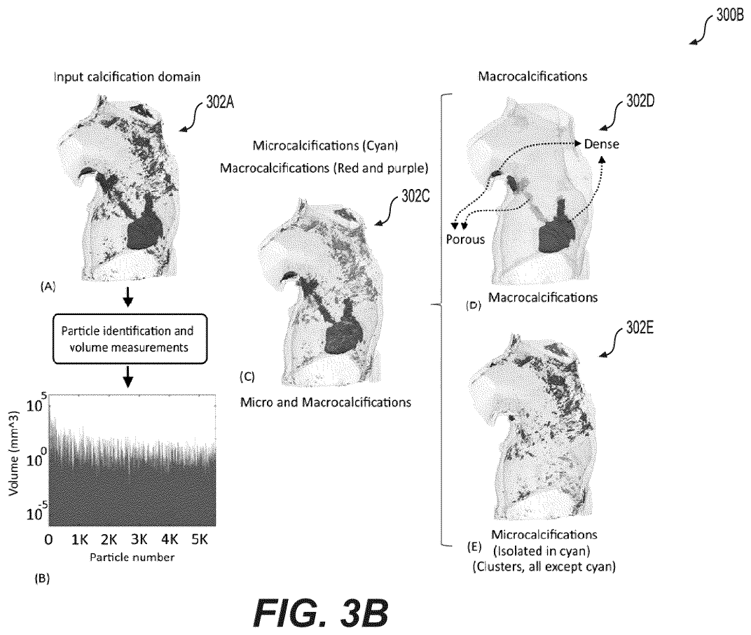

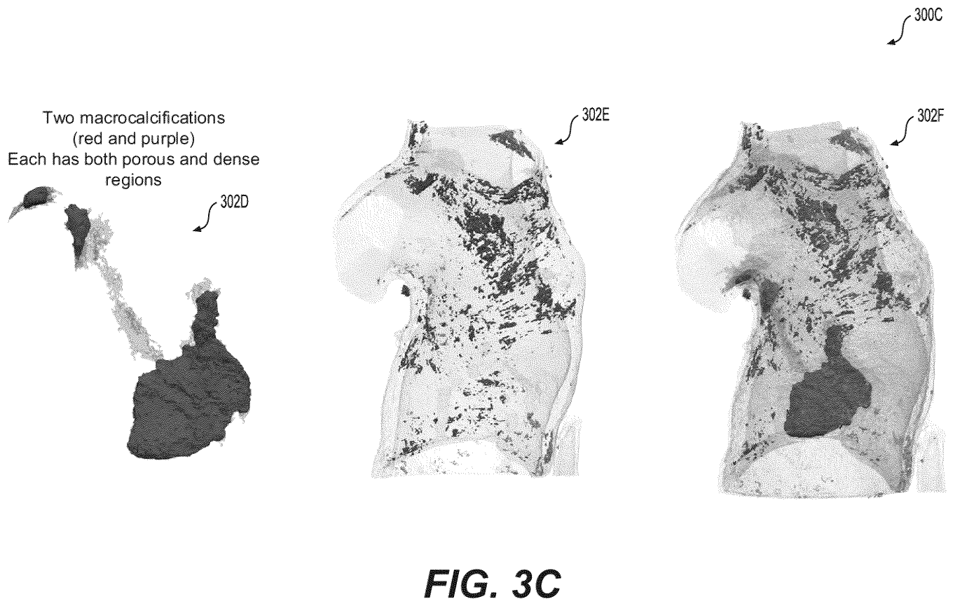

FIGS. 3A-3C illustrate one or more example renderings 300A, 300B, and 300C for identifying, quantifying, and phenotyping vascular calcification (VC) in one or more patients, in accordance with the presently disclosed embodiments. For example, as depicted by FIG. 3A, the identification and quantification of vascular calcifications (e.g., depicted as reddish portions), lipid pool regions (e.g., depicted as yellowish portions), and tissue regions (e.g., depicted as grayish portions) are shown for each of a number of arterial samples 302, 304, and 306 and each of a number of aneurysm samples 308 and 310. Similarly, as depicted by FIGS. 3B and 3C, quantification of vascular calcifications based on size, spatial distribution, topology, porosity (sparsity), and lipid pool colocalization are shown. For example, example renderings 302C depicts that vascular calcifications classified by size may be classified as microcalcifications or macrocalcifications. Similarly, example renderings 302E and 302F depict that microcalcifications may be further classified as isolated or as part of cluster. Example renderings 302A and 302D depict that the volume of each of the identified macrocalcifications may be further classified as sparse or dense.

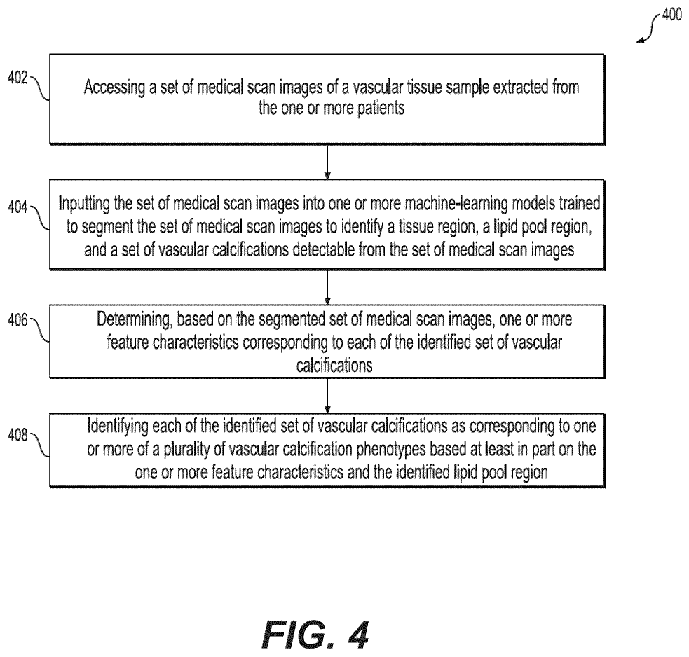

FIG. 4 illustrates a flow diagram of a method 400 for identifying, quantifying, and phenotyping vascular calcification (VC) in one or more patients, in accordance with the disclosed embodiments. The method 400 may be performed utilizing one or more processing devices 114 that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other artificial intelligence (AI)/machine-learning (ML) accelerators device(s) that may be suitable for processing medical data and making one or more predictions or decisions based thereon), firmware (e.g., microcode), or some combination thereof.

The method 400 may begin at block 402 with the one or more processing devices 114 accessing a set of medical scan images of a vascular tissue sample extracted from one or more patients. For example, the one or more processing devices 114 may receive one or more μ-CT scans (e.g., medical scan images 119) of a vascular tissue sample 108A-108D extracted from one or more patients 102A-102D. The method 400 may then continue at block 404 with the one or more processing devices 114 inputting the set of medical scan images into one or more machine-learning models trained to segment the set of medical scan images to identify a tissue region, a lipid pool region, and a set of vascular calcifications detectable from the set of medical scan images. For example, in some embodiments, one or more μ-CT scans (e.g., medical scan images 119) may be inputted into one or more machine-learning models 118 (e.g., machine-learning pipeline including one or more semantic segmentation models, classification models, and clustering models), which may generate a segmentation map including an identified tissue region, an identified lipid pool region, and an identified set of vascular calcifications.

The method 400 may then continue at block 406 with the one or more processing devices 114 determining, based on the segmented set of medical scan images, one or more feature characteristics corresponding to each of the identified set of vascular calcifications. For example, in some embodiments, the one or more machine-learning models 118 (e.g., machine-learning pipeline including one or more semantic segmentation models, classification models, and clustering models) may feature characteristics corresponding to each of the identified set of vascular calcifications including, for example, size (e.g., macrocalcifications vs. microcalcifications), spatial distribution of microcalcifications (e.g., clustered microcalcifications vs. isolated microcalcifications), topology of macrocalcifications (e.g., sparse macrocalcifications vs. dense macrocalcifications), and co-localization with lipid pools (e.g., atherosclerotic vascular calcifications vs. non-atherosclerotic vascular calcifications).

The method 400 may then conclude at block 408 with the one or more processing devices 114 identifying each of the identified set of vascular calcifications as corresponding to one or more of a plurality of vascular calcification phenotypes based at least in part on the one or more feature characteristics and the identified lipid pool region. For example, in some embodiments, the one or more machine-learning models 118 (e.g., machine-learning pipeline including one or more semantic segmentation models, classification models, and clustering models) may generate predictions of vascular calcification phenotypes 120 for each of the identified set of vascular calcifications. In certain embodiments, the predictions of vascular calcification phenotypes 120 for each of the identified set of vascular calcifications may include predictions of a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall (e.g., innermost layer, middle layer, and outermost layer).

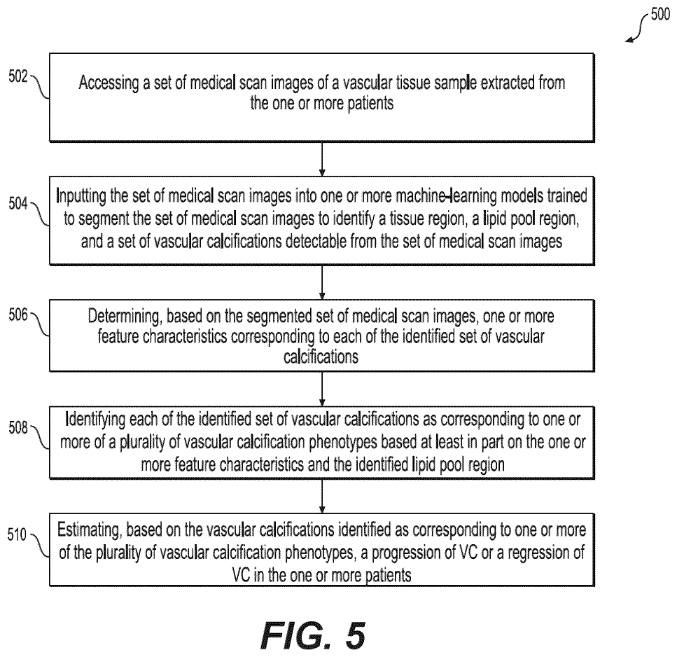

FIG. 5 illustrates a flow diagram of a method 500 for estimating a progression of vascular calcification (VC) or regression of VC in one or more patients, in accordance with the presently disclosed embodiments. The method 500 may be performed utilizing one or more processing devices 114 that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other artificial intelligence (AI)/machine-learning (ML) accelerators device(s) that may be suitable for processing medical data and making one or more predictions or decisions based thereon), firmware (e.g., microcode), or some combination thereof.

The method 500 may begin at block 502 with the one or more processing devices 114 accessing a set of medical scan images of a vascular tissue sample extracted from one or more patients. For example, the one or more processing devices 114 may receive one or more μ-CT scans (e.g., medical scan images 119) of a vascular tissue sample 108A-108D extracted from one or more patients 102A-102D. The method 500 may then continue at block 504 with the one or more processing devices 114 inputting the set of medical scan images into one or more machine-learning models trained to segment the set of medical scan images to identify a tissue region, a lipid pool region, and a set of vascular calcifications detectable from the set of medical scan images. For example, in some embodiments, one or more μ-CT scans (e.g., medical scan images 119) may be inputted into one or more machine-learning models 118 (e.g., machine-learning pipeline including one or more semantic segmentation models, classification models, and clustering models), which may generate a segmentation map including an identified tissue region, an identified lipid pool region, and an identified set of vascular calcifications.

The method 500 may then continue at block 506 with the one or more processing devices 114 determining, based on the segmented set of medical scan images, one or more feature characteristics corresponding to each of the identified set of vascular calcifications. For example, in some embodiments, the one or more machine-learning models 118 (e.g., machine-learning pipeline including one or more semantic segmentation models, classification models, and clustering models) may feature characteristics corresponding to each of the identified set of vascular calcifications including, for example, size (e.g., macrocalcifications vs. microcalcifications), spatial distribution of microcalcifications (e.g., clustered microcalcifications vs. isolated microcalcifications), topology of macrocalcifications (e.g., sparse macrocalcifications vs. dense macrocalcifications), and co-localization with lipid pools (e.g., atherosclerotic vascular calcifications vs. non-atherosclerotic vascular calcifications).

The method 500 may then continue at block 508 with the one or more processing devices 114 identifying each of the identified set of vascular calcifications as corresponding to one or more of a plurality of vascular calcification phenotypes based at least in part on the one or more feature characteristics and the identified lipid pool region. For example, in some embodiments, the one or more machine-learning models 118 (e.g., machine-learning pipeline including one or more semantic segmentation models, classification models, and clustering models) may generate predictions of vascular calcification phenotypes 120 for each of the identified set of vascular calcifications. In certain embodiments, the predictions of vascular calcification phenotypes 120 for each of the identified set of vascular calcifications may include predictions of a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall (e.g., innermost layer, middle layer, and outermost layer).

The method 500 may then conclude at block 510 with the one or more processing devices 114 estimating, based on the vascular calcifications identified as corresponding to one or more of the plurality of vascular calcification phenotypes, a progression of VC or a regression of VC in the one or more patients. For example, in some embodiments, the one or more machine-learning models 118 (e.g., machine-learning pipeline including one or more semantic segmentation models, classification models, and clustering models) may utilize the predictions of the macrocalcification phenotype, the microcalcification phenotype, the sparse calcification phenotype, the dense calcification phenotype, the clustered calcification phenotype, the isolated calcification phenotype, the atherosclerotic phenotype, the non-atherosclerotic phenotype, and the phenotype identifying a location of vascular calcifications within an arterial wall (e.g., innermost layer, middle layer, and outermost layer) to determine whether identified set of vascular calcifications corresponds to one of a protective vascular calcification, in which one or more particular vascular calcifications are of a healthful quality, or a destabilizing vascular calcification, in which one or more particular vascular calcifications are of a deleterious quality.

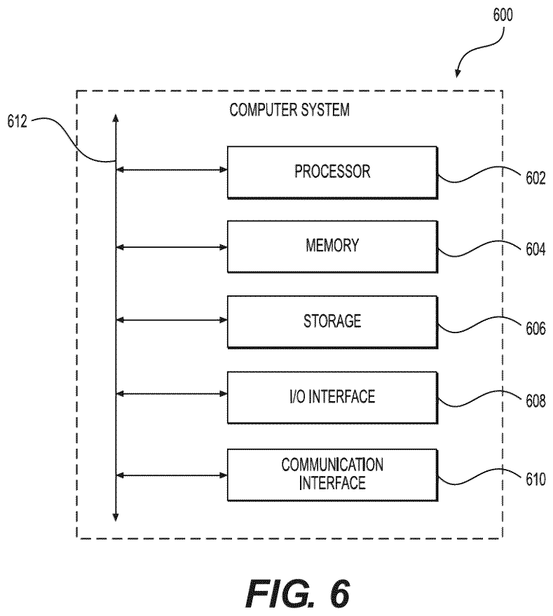

FIG. 6 illustrates an example of one or more computing device(s) 600 that may be utilized for identifying, quantifying, and phenotyping vascular calcification (VC) in one or more patients, in accordance with the presently disclosed embodiments. In certain embodiments, the one or more computing device(s) 600 may perform one or more steps of one or more methods described or illustrated herein. In certain embodiments, the one or more computing device(s) 600 provide functionality described or illustrated herein. In certain embodiments, software running on the one or more computing device(s) 600 performs one or more steps of one or more methods described or illustrated herein, or provides functionality described or illustrated herein. Certain embodiments include one or more portions of the one or more computing device(s) 600.

This disclosure contemplates any suitable number of computing systems 600. This disclosure contemplates one or more computing device(s) 600 taking any suitable physical form. As example and not by way of limitation, one or more computing device(s) 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 600 may be unitary or distributed; span multiple locations; span multiple machines; span multiple data centers; or reside in a cloud, which may include one or more cloud components in one or more networks.

Where appropriate, the one or more computing device(s) 600 may perform without substantial spatial or temporal limitation one or more steps of one or more methods described or illustrated herein. As an example, and not by way of limitation, the one or more computing device(s) 600 may perform, in real-time or in batch mode, one or more steps of one or more methods described or illustrated herein. The one or more computing device(s) 600 may perform, at different times or at different locations, one or more steps of one or more methods described or illustrated herein, where appropriate.

In certain embodiments, the one or more computing device(s) 600 includes a processor 602, memory 604, database 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or database 606; decode and execute them; and then write one or more results to an internal register, an internal cache, memory 604, or database 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or database 606, and the instruction caches may speed up retrieval of those instructions by processor 602.

Data in the data caches may be copies of data in memory 604 or database 606 for instructions executing at processor 602 to operate on; the results of previous instructions executed at processor 602 for access by subsequent instructions executing at processor 602 or for writing to memory 604 or database 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, the one or more computing device(s) 600 may load instructions from database 606 or another source (such as, for example, another one or more computing device(s) 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604.

In certain embodiments, processor 602 executes only instructions in one or more internal registers, internal caches, or memory 604 (as opposed to database 606 or elsewhere) and operates only on data in one or more internal registers, internal caches, or memory 604 (as opposed to database 606 or elsewhere). One or more memory buses (which may each include an address bus and a data bus) may couple processor 602 to memory 604. Bus 612 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM

(DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, database 606 includes mass storage for data or instructions. As an example, and not by way of limitation, database 606 may include a hard disk drive (HDD), a floppy disk drive, flash memory, an optical disc, a magneto-optical disc, magnetic tape, or a Universal Serial Bus (USB) drive, or a combination of two or more of these. Database 606 may include removable or non-removable (or fixed) media, where appropriate. Database 606 may be internal or external to the one or more computing device(s) 600, where appropriate. In certain embodiments, database 606 is non-volatile, solid-state memory. In certain embodiments, database 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), flash memory, or a combination of two or more of these. This disclosure contemplates mass database 606 taking any suitable physical form. Database 606 may include one or more storage control units facilitating communication between processor 602 and database 606, where appropriate. Where appropriate, database 606 may include one or more databases 606. Although this disclosure describes and illustrates particular storage, this disclosure contemplates any suitable storage.

In certain embodiments, I/O interface 608 includes hardware, software, or both, providing one or more interfaces for communication between the one or more computing device(s) 600 and one or more I/O devices. The one or more computing device(s) 600 may include one or more of these I/O devices, where appropriate. One or more of these I/O devices may enable communication between a person and the one or more computing device(s) 600. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between the one or more computing device(s) 600 and one or more other computing device(s) 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it.

As an example, and not by way of limitation, the one or more computing device(s) 600 may communicate with an ad hoc network, a personal area network (PAN), a local area network (LAN), a wide area network (WAN), a metropolitan area network (MAN), one or more portions of the Internet, or a combination of two or more of these. One or more portions of one or more of these networks may be wired or wireless. As an example, the one or more computing device(s) 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), other suitable wireless network, or a combination of two or more of these. The one or more computing device(s) 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of the one or more computing device(s) 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, another suitable bus, or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

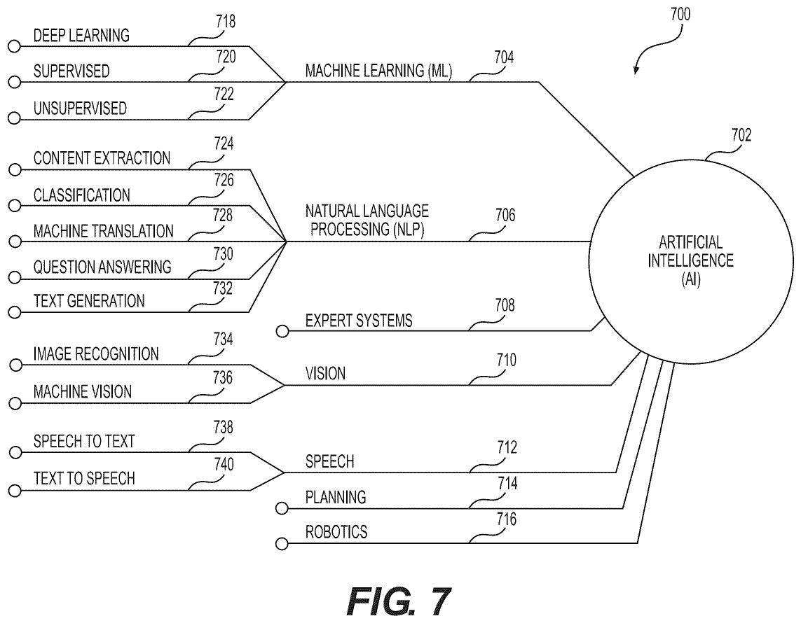

FIG. 7 illustrates a diagram 700 of an example artificial intelligence (AI) architecture 702 (which may be included as part of the one or more computing device(s) 600 as discussed above with respect to FIG. 6) that may be utilized for identifying, quantifying, and phenotyping vascular calcification (VC) in one or more patients, in accordance with the presently disclosed embodiments. In certain embodiments, the AI architecture 702 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other artificial intelligence (AI)/machine-learning (ML) accelerator device(s) that may be suitable for processing various data and making one or more predictions or decisions based thereon), software (e.g., instructions running/executing on one or more processing devices), firmware (e.g., microcode), or some combination thereof.

In certain embodiments, as depicted by FIG. 7, the AI architecture 702 may include machine learning (ML) models 704, natural language processing (NLP) models 706, expert systems 708, computer-based vision models 710, speech recognition models 712, planning models 714, and robotics models 716. In certain embodiments, the ML models 704 may include any statistics-based models that may be suitable for finding patterns across large amounts of data (e.g., “Big Data” such as genomics data, proteomics data, metabolomics data, metagenomics data, transcriptomics data, or other omics data). For example, in certain embodiments, the ML models 704 may include deep learning models 718, supervised learning models 720, and unsupervised learning models 722.

In certain embodiments, the deep learning models 718 may include any artificial neural networks (ANNs) that may be utilized to learn deep levels of representations and abstractions from large amounts of data. For example, the deep learning models 718 may include ANNs, such as a perceptron, a multilayer perceptron (MLP), an autoencoder (AE), a convolution neural network (CNN), a recurrent neural network (RNN), long short term memory (LSTM), a gated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning models 720 may include any algorithms that may be utilized to apply, for example, what has been learned in the past to new data using labeled examples for predicting future events. For example, starting from the analysis of a known training data set, the supervised learning models 720 may produce an inferred function to make predictions about the output values. The supervised learning models 720 may also compare its output with the correct and intended output and find errors in order to modify the supervised learning models 720 accordingly. On the other hand, the unsupervised learning models 722 may include any algorithms that may be applied, for example, when the data used to train the unsupervised learning models 722 are neither classified nor labeled. For example, the unsupervised learning models 722 may study and analyze how systems may infer a function to describe a hidden structure from unlabeled data.

In certain embodiments, the NLP models 706 may include any algorithms or functions that may be suitable for automatically manipulating natural language, such as speech and/or text. For example, in some embodiments, the NLP models 706 may include content extraction models 724, classification models 726, machine translation models 728, question answering (QA) models 730, and text generation models 732. In certain embodiments, the content extraction models 724 may include a means for extracting text or images from electronic documents (e.g., webpages, text editor documents, and so forth) to be utilized, for example, in other applications.

In certain embodiments, the classification models 726 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation models 728 may include any algorithms or functions that may be suitable for automatically converting source text in one language, for example, into text in another language. The QA models 730 may include any algorithms or functions that may be suitable for automatically answering questions posed by humans in, for example, a natural language, such as that performed by voice-controlled personal assistant devices. The text generation models 732 may include any algorithms or functions that may be suitable for automatically generating natural language texts.

In certain embodiments, the expert systems 708 may include any algorithms or functions that may be suitable for simulating the judgment and behavior of a human or an organization that has expert knowledge and experience in a particular field (e.g., stock trading, medicine, sports statistics, and so forth). The computer-based vision models 710 may include any algorithms or functions that may be suitable for automatically extracting information from images (e.g., photo images, video images). For example, the computer-based vision models 710 may include image recognition algorithms 734 and machine vision algorithms 736. The image recognition algorithms 734 may include any algorithms that may be suitable for automatically identifying and/or classifying objects, places, people, and so forth that may be included in, for example, one or more image frames or other displayed data. The machine vision algorithms 736 may include any algorithms that may be suitable for allowing computers to “see”, or, for example, to rely on image sensors or cameras with specialized optics to acquire images for processing, analyzing, and/or measuring various data characteristics for decision making purposes.

In certain embodiments, the speech recognition models 712 may include any algorithms or functions that may be suitable for recognizing and translating spoken language into text, such as through automatic speech recognition (ASR), computer speech recognition, speech-to-text (STT) 738, or text-to-speech (TTS) 740 in order for the computing to communicate via speech with one or more users, for example. In certain embodiments, the planning models 714 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics models 716 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, may be claimed in another claim category, e.g. system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

What is claimed is:

1. A method for identifying, quantifying, and phenotyping vascular calcification (VC) in one or more patients, comprising, by one or more computing devices:
accessing a set of medical scan images of a vascular tissue sample extracted from one or more patients;
inputting the set of medical scan images into one or more machine-learning models trained to segment the set of medical scan images to identify a tissue region, a lipid pool region, and a set of vascular calcifications detectable from the set of medical scan images;
determining, based on the segmented set of medical scan images, one or more feature characteristics corresponding to each of the identified set of vascular calcifications; and
identifying each of the identified set of vascular calcifications as corresponding to one or more of a plurality of vascular calcification phenotypes based at least in part on the one or more feature characteristics and the identified lipid pool region.

2. The method of claim 1, wherein the plurality of vascular calcification phenotypes comprises a macrocalcification phenotype, a microcalcification phenotype, a sparse calcification phenotype, a dense calcification phenotype, a clustered calcification phenotype, an isolated calcification phenotype, an atherosclerotic phenotype, a non-atherosclerotic phenotype, and a phenotype identifying a location of vascular calcifications within an arterial wall of a vasculature of the one or more patients.

3. The method of claim 1, wherein identifying each of the identified set of vascular calcifications as corresponding to one or more of the plurality of vascular calcification phenotypes comprises identifying one or more biomarkers associated with a risk for a major adverse cardiovascular event (MACE).

4. The method of claim 1, wherein identifying each of the identified set of vascular calcifications as corresponding to one or more of the plurality of vascular calcification phenotypes comprises identifying a risk for a major adverse cardiovascular event (MACE) for the one or more patients.

5. The method of claim 1, wherein identifying each of the identified set of vascular calcifications as corresponding to one or more of the plurality of vascular calcification phenotypes comprises identifying each of the identified set of vascular calcifications as corresponding to one of a destabilizing vascular calcification or a protective vascular calcification.

6. The method of claim 1, further comprising identifying each of the identified set of vascular calcifications as corresponding to at least one of the plurality of vascular calcification phenotypes based at least in part on whether each of the identified set of vascular calcifications is associated with the identified lipid pool region.

7. The method of claim 1, wherein determining the one or more feature characteristics comprises determining one or more of a size, a spatial distribution, a topology, a porosity, or a lipid pool colocalization for each of the identified set of vascular calcifications.

8. The method of claim 1, wherein the one or more machine-learning models comprise a semantic segmentation model and at least one classification model.

9. The method of claim 8, further comprising:
training the semantic segmentation model by:
accessing a data set of medical scan images of one or more vascular tissue samples extracted from a plurality of patients, wherein the data set of medical scan images comprises sparse annotations of tissue regions and lipid pool regions in the one or more vascular tissue samples;
partitioning the data set of medical scan images into a model-training data set and a model-validation data set;
training, based on the model-training data set, the semantic segmentation model to generate a segmentation map comprising a first set of pixels corresponding to tissue regions and a second set of pixels corresponding to background regions; and
evaluating the semantic segmentation model based on the model-validation data set.

10. The method of claim 8, wherein the at least one classification model comprises a first classification model, the method further comprising:
training the first classification model by:
accessing a segmentation map generated by the semantic segmentation model, a set of pixel spatial information, and a set of pixel color intensity information associated with the set of medical scan images;
flattening the segmentation map into a feature map; and
inputting the feature map, the set of pixel spatial information, and the set of pixel color intensity information into the first classification model to identify one or more intra-slice tissue regions detectable at least in part from the first set of pixels corresponding to tissue regions in the segmentation map; and

11. The method of claim 10, wherein the one or more machine-learning models comprise a second classification model associated with the first classification model, the method further comprising:
training the second classification model by:
manual extraction of feature maps from the input images by passing them through convolutional layers with known weights;
flattening the segmentation maps into one-dimensional feature maps;
extracting one or more pixels of the flattened one-dimensional feature maps corresponding to tissue regions by passing the one-dimensional feature maps through a foreground-pass filter;
inputting the extracted one or more pixels and the set of pixel spatial information into the second classification model to generate a prediction of a class label for one or more lipid pool regions detectable at least in part from the extracted one or more pixels and the set of pixel spatial information; and
outputting, by the second classification model, the prediction of the class label for the one or more lipid pool regions.

12. The method of claim 1, wherein the set of medical scan images comprises one or more micro computed tomography (μ-CT) images, one or more micro positron emission tomography (μ-PET) images, one or more micron single-photon emission computed tomography (u-SPECT) images, one or more optical coherence tomography (OCT) images, one or more optical coherence tomography angiography (OCT-A) images, or any combination thereof.

13. The method of claim 1, wherein the set of medical scans comprises a first set of medical scans of a first vascular tissue sample extracted from the one or more patients at an initial date, the method further comprising:
accessing a second set of medical scan images of a second vascular tissue sample extracted from the one or more patients, the second vascular tissue sample being extracted from the one or more patients at a date subsequent to the initial date;
inputting the second set of medical scan images into the one or more machine-learning models trained to segment the second set of medical scan images to identify a second tissue region, a second lipid pool region, and a second set of vascular calcifications detectable from the second set of medical scan images;
determining, based on the segmented second set of medical scan images, one or more second featural characteristics corresponding to each of the identified second set of vascular calcifications;
identifying each of the identified second set of vascular calcifications as corresponding to one or more of the plurality of vascular calcification phenotypes based at least in part on the one or more second featural characteristics and the identified second lipid pool region; and
estimating, based on the vascular calcifications identified as corresponding to one or more of the plurality of vascular calcification phenotypes, a progression of VC or a regression of VC in the one or more patients.

14. The method of claim 13, further comprising identifying an effective treatment regimen of one or more calcification inhibitors to treat the one or more patients based on the estimated progression of VC or regression of VC.

15. The method of claim 13, wherein the date subsequent to the initial date comprises one or more dates selected from the group comprising approximately 0.25 months, 0.5 months, 0.75 months, 1 month, 3 months, 6 months, 9 months, 12 months, 15 months, 18 months, 21 months, 24 months, 27 months, 30 months, 33 months, 36 months, 39 months, 42 months, 45 months, or 48 months from the initial date.

16. The method of claim 1, further comprising estimating, based on the vascular calcifications identified as corresponding to one or more of the plurality of vascular calcification phenotypes, whether the one or more patients is responsive to a treatment.

17. The method of claim 14, wherein the treatment comprises one or more calcification inhibitors.

18. The method of claim 1, further comprising:
generating a report based on the vascular calcifications of the identified set of vascular calcifications identified as corresponding to one or more of the plurality of vascular calcification phenotypes; and
transmitting the report to a computing device associated with a clinician or a scientist.

19. A system including one or more computing devices for identifying, quantifying, and phenotyping vascular calcification (VC) in one or more patients, the one or more computing devices comprising:
one or more non-transitory computer-readable storage media including instructions; and
one or more processors coupled to the one or more storage media, the one or more processors configured to execute the instructions to perform operations comprising:
accessing a set of medical scan images of a vascular tissue sample extracted from the one or more patients;
inputting the set of medical scan images into one or more machine-learning models trained to segment the set of medical scan images to identify a tissue region, a lipid pool region, and a set of vascular calcifications detectable from the set of medical scan images;
determining, based on the segmented set of medical scan images, one or more featural characteristics corresponding to each of the identified set of vascular calcifications; and
identifying each of the identified set of vascular calcifications as corresponding to one or more of a plurality of vascular calcification phenotypes based at least in part on the one or more featural characteristics and the identified lipid pool region.

20. A non-transitory computer-readable medium comprising instructions that, when executed by one or more processors of one or more computing devices, cause the one or more processors to execute the instructions to perform operations comprising:
accessing a set of medical scan images of a vascular tissue sample extracted from one or more patients;
inputting the set of medical scan images into one or more machine-learning models trained to segment the set of medical scan images to identify a tissue region, a lipid pool region, and a set of vascular calcifications detectable from the set of medical scan images;
determining, based on the segmented set of medical scan images, one or more featural characteristics corresponding to each of the identified set of vascular calcifications; and
identifying each of the identified set of vascular calcifications as corresponding to one or more of a plurality of vascular calcification phenotypes based at least in part on the one or more featural characteristics and the identified lipid pool region.
